SEQUENCE LISTING (2 sequences)

SEQ ID NO: 1 - DNA, 21 bp, synthetic construct
  source 1..21: mol_type = other DNA
cggtgtcctggtgttcttgtc

SEQ ID NO: 2 - DNA, 21 bp, synthetic construct
  source 1..21: mol_type = other DNA
tggagtatttgggtgcatttc

(19) United States
(12) Patent Application Publication
(10) Pub. No.: US 2025/0346965 A1
(43) Pub. Date: Nov. 13, 2025

(54) PRIMER FOR AMPLIFYING CLOSELY LINKED MOLECULAR MARKER OF POWDERY MILDEW RESISTANCE GENE PmDR739 OF TRITICUM DICOCCOIDES AND APPLICATION THEREOF

(71) Applicants: Crop Research Institute, Shandong Academy of Agricultural Sciences, Jinan (CN); Yantai University, Yantai (CN); National Center of Technology Innovation for Comprehensive Utilization of Saline-Alkali Land, Dongying (CN)

(72) Inventors: Cheng LIU, Jinan (CN); Yuli JIN, Yantai (CN); Pengtao MA, Yantai (CN); Wenjing XU, Jinan (CN); Ran HAN, Jinan (CN); Dungong CHENG, Jinan (CN); Yan ZI, Jinan (CN); Faji LI, Jinan (CN); Aifeng LIU, Jinan (CN); Haosheng LI, Jinan (CN)

(21) Appl. No.: 19/015,811

(22) Filed: Jan. 10, 2025

(30) Foreign Application Priority Data
May 10, 2024 (CN) 2024105757613

(51) Int. Cl. C12Q 1/6895; C12Q 1/686

(52) U.S. Cl. CPC C12Q 1/6895; C12Q 1/686; C12Q 2600/13

(57) ABSTRACT

Disclosed is a primer for amplifying a closely linked molecular marker of a powdery mildew resistance gene PmDR739 of Triticum dicoccoides. The molecular marker primer includes a forward primer HENU686-F and a reverse primer HENU686-R, where the nucleotide sequence of the forward primer HENU686-F is shown as SEQ ID NO:1, and the nucleotide sequence of the reverse primer HENU686-R is shown as SEQ ID NO:2. The molecular marker primer provided by the present disclosure can be applied in detection and identification of the gene PmDR739, auxiliary identification of a powdery mildew resistance trait of wheat, and molecular marker-assisted selection breeding.

### TECHNICAL FIELD

The present disclosure relates to the technical field of agricultural biological genes, and in particular to a primer for amplifying a closely linked molecular marker of a powdery mildew resistance gene PmDR739 of Triticum dicoccoides and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 25005TLAN-USP1-SL.xml. The XML file is 2,936 bytes; is created on Jan. 9, 2025; and is being submitted electronically via patent center.

### BACKGROUND

Wheat is a worldwide food, providing about 21% of food calories and 20% of protein for human beings. It is also the third largest food crop in China (Shiferaw et al. Crops that feed the world 10. Past successes and future challenges to the role played by wheat in global food security. Food Security, 2013, 5:291-317). Wheat powdery mildew is one of main disease in wheat production. Measures to control the wheat powdery mildew mainly include chemical control and breeding and popularization of resistance varieties. Compared with the traditional chemical control method, the method of digging wheat varieties with resistance genes and breeding disease-resistant varieties by using resistance sources and resistance genes is a safe, economic and effective method for controlling the wheat powdery mildew.

Up to now, 69 powdery mildew resistance genes (Pm1-Pm69) of wheat have been officially named, and different named genes may also come from the same locus, such as Pm3=Pm8=Pm17, Pm18=Pm1c, Pm22=Pm1e, Pm23=Pm4c, and Pm31=Pm21 (Wang et al. Fighting wheat powdery mildew: from genes to fields. Theoretical and applied genetics, 2023, 136:27). Since most of the disease-resistant genes are the major genes of pathogen race specialization, long-term and large-scale utilization of a single resistance source will accelerate the variation of pathogen races and lead to the loss of resistance of the disease-resistant genes. In China, the resistance of the genes including Pm1, Pm2, Pm3a, Pm3c, Pm3f, Pm4b, Pm5, Pm6, Pm7, Pm8, etc. has been gradually lost. Disease resistance of wheat can be significantly improved through pyramiding of a plurality of resistance genes, for example, the resistance of Pm2+Pm4b and Pm2+Pm6 is significantly higher than that of a single resistance gene (He et al. Research progress and prospect of adult plant resistance to stripe rust and powdery mildew in wheat. Agricultural Sciences in China, 2011, 44:2193-2215).

Relative species of wheat contain a large number of excellent genes, which can enrich the genetic diversity of the wheat. Triticum dicoccoides (2n=28, AABB) is the tetraploid ancestor of common wheat, belonging to the secondary gene pool of the common wheat. At present, the powdery mildew resistance genes discovered from the Triticum dicoccoides include Pm16, Pm26, Pm30, Pm36, Pm41, Pm42, Pm64 and Pm69. There is a chromosome complement homologous with the wheat in the Triticum dicoccoides, which can realize the transfer of beneficial genes into wheat by pairing exchange between homologous chromosomes. Compared with Triticum dicoccum, the genome genetic information of the Triticum dicoccoides is more abundant and has more research significance (Avni et al. Wild emmer genome architecture and diversity elucidate wheat evolution and domestication. Science, 2017, 357:93-97). Therefore, it is of great significance to find new powdery mildew resistance genes from the Triticum dicoccoides, a relative species of the wheat, for rational utilization of resistance sources, acceleration of gene polymerization breeding and broadening of genetic basis of the wheat.

### SUMMARY

An objective of the present disclosure is to provide a primer for amplifying a closely linked molecular marker of a powdery mildew resistance gene PmDR739 of Triticum dicoccoides and an application thereof, so as to map and detect the powdery mildew resistance gene PmDR739 of Triticum dicoccoides by utilizing the primer-amplified molecular marker, purposefully select parents during wheat breeding, and provide guidance basis for selection of powdery mildew-resistant new varieties of wheat.

The present disclosure is implemented through the following method: a primer for amplifying a closely linked molecular marker of a powdery mildew resistance gene PmDR739 of Triticum dicoccoides, where the molecular marker is a codominant SSR marker HENU686, and the primer includes a forward primer HENU686-F and a reverse primer HENU686-R.

A forward primer of the molecular marker HENU686 is HENU686-F, and the nucleotide sequence is:

5′-CGGTGTCCTGGTGTTCTTGTC-3′, as shown in SEQ
ID NO: 1;

and
a reverse primer of the molecular marker HENU686 is HENU686-R, and the nucleotide sequence is:

5′-TGGAGTATTTGGGTGCATTTC-3′, as shown in SEQ
ID NO: 2.

The present disclosure provides an application of the primer for amplifying a closely linked molecular marker of a powdery mildew resistance gene PmDR739 of Triticum dicoccoides in detection and identification of the gene PmDR739, auxiliary identification of a powdery mildew resistance trait of wheat, and molecular marker-assisted selection breeding.

The present disclosure further provides a method for detecting whether a wheat sample carries a powdery mildew resistance gene PmDR739 of Triticum dicoccoides. The method includes the following steps:
(1) extracting a genomic deoxyribonucleic acid (DNA) of a wheat sample to be detected;
(2) performing polymerase chain reaction (PCR) amplification on the genomic DNA of the wheat sample to be detected by utilizing a molecular marker primer to obtain an amplification product, where the molecular marker primer includes a forward primer HENU686-F and a reverse primer HENU686-R,
a forward primer of the molecular marker HENU686 is HENU686-F, the nucleotide sequence is:

5′-CGGTGTCCTGGTGTTCTTGTC-3′, as shown in SEQ
ID NO: 1;

a reverse primer of the molecular marker HENU686 is HENU686-R, and the nucleotide sequence is:

5′-TGGAGTATTTGGGTGCATTTC-3′, as shown in SEQ
ID NO: 2.

and
(3) performing electrophoresis and detection on the amplification product, where if two specific bands of 280 bp and 370 bp are capable of being simultaneously amplified, the wheat sample to be detected is indicated to carry the powdery mildew resistance gene PmDR739 of Triticum dicoccoides, otherwise, the wheat sample to be detected is indicated not to carry the powdery mildew resistance gene PmDR739 of Triticum dicoccoides.

A PCR amplification system in step (2) is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification in step (2) is: performing predenaturation for 3 minutes at 94° C., performing denaturation for 15 seconds at 94° C., performing annealing for 20 seconds at 55° C., performing extension for 40 seconds, and performing 30 cycles; performing extension for 10 minutes at 72° C.; and performing preservation at 4° C.

A procedure of the electrophoresis of the amplification product in step (3) is: performing electrophoresis on a non-denaturing polyacrylamide gel with the mass volume percentage concentration of 8%, after the amplification product is mixed with 2 μL of 6×sample loading buffer, taking 2 μL of the mixture for spot sampling, performing electrophoresis under constant pressure of 180 V for 2.5-3 hours, and performing photographing after silver nitrate staining.

The Triticum dicoccoides DR739 used in the research of the present disclosure has high powdery mildew resistance in the seedling stage and an adult plant stage, and genetic analysis and molecular marker detection of powdery mildew resistance in the seedling stage show that the resistance of the Triticum dicoccoides DR739 to wheat powdery mildew with different toxicity in the seedling stage is controlled by a pair of dominant genes PmDR739, which are mapped on a wheat chromosome 2AL, and are a novel powdery mildew resistance gene/allele of wheat. Through genetic segregation population detection, the molecular marker HENU686 of the powdery mildew resistance gene PmDR739 of wheat provided by the present disclosure is found to have a genetic distance of 0.85 cM from the gene PmDR739, and be closely linked to the gene PmDR739, which can be used for accurately detecting a large genetic mapping population of the gene PmDR739 and applied to fine mapping and map-based cloning of the gene PmDR739.

The present disclosure provides the primer for amplifying a closely linked molecular marker of a powdery mildew resistance gene PmDR739 of wheat and the application thereof, such that target varieties are quickly and accurately screened, which is not affected by an environment, the selection target is clear, the production cost is saved, and the selection efficiency and quality of high-quality powdery mildew-resistant wheat varieties or strains are greatly improved.

### BRIEF DESCRIPTION OF THE DRAWING

FIG. 1 shows some results of detection of Triticum dicoccoides DR739 and Triticum turgidum Langdon as well as segregation populations of hybrids thereof through a primer of a molecular marker HENU686.

In the figure, M: DL2000; 1: Triticum dicoccoides DR739 (powdery mildew resistance variety carrying the gene PmDR739); 2: Triticum turgidum Langdon (powdery mildew susceptible variety); 3-22: F_{2:3} families formed by crossing the Triticum dicoccoides DR739 with the Triticum turgidum Langdon, where 3-7: F_{2:3} family homozygous for resistance, 8-17: F_{2:3} family for resistance and susceptibility segregation, and 18-22: F_{2:3} family homozygous for susceptibility; and bands indicated by two white arrows are specific bands tracing the gene PmDR739.

### DETAILED DESCRIPTION OF THE EMBODIMENTS

The following embodiments are used for understanding the present disclosure better, but not intended to limit the present disclosure. Experimental methods in the following embodiments are conventional methods unless otherwise specified. All experimental materials, reagents, etc. used in the embodiments may be obtained from a commercial approach unless otherwise specified.

Embodiment 1 Development of molecular marker HENU686 primer of powdery mildew resistance gene PmDR739 of wheat

### 1. Materials

The resistance parent Triticum dicoccoides DR739 and the susceptible parent Triticum turgidum Langdon are resistant and susceptible to wheat powdery mildew respectively. F₁ hybrids of the Triticum dicoccoides DR739 and the Triticum turgidum Langdon are selfed to obtain a F₂ population and corresponding F_{2:3} families.

### 2. Genomic DNA Extraction of Wheat

A cetyltrimethylammonium bromide (CTAB) method for genomic DNA extraction of wheat includes the process as follows:
(1) Fresh wheat leaves are cut and ground with liquid nitrogen about 0.5 g of ground wheat leaves is put into a 2.0 mL centrifuge tube.
(2) 600 μL of CTAB extract solution is added and placed in a water bath at 65° C. for 40 minutes, where mixing up and down is performed once every 10 minutes.
(3) 600 μL of chloroform-isoamyl alcohol (24:1, v/v) is added and shaken gently on a shaking table for 15 minutes.
(4) Centrifugation is performed at 12000 rpm for 15 minutes, supernatant is taken to another 2 mL centrifuge tube, 3 times volume of precooled absolute ethanol is added, and precipitation is performed at a refrigerator at −20° C. for 60 minutes.
(5) Flocculent DNA precipitate is picked out and washed twice with 75% precooled ethanol.
(6) The DNA precipitate is picked out, placed in a 1.5 mL centrifuge tube, and air-dried indoors.
(7) 60 μL of TE buffer (100 mM Tris-Hcl, 10 mM EDTA, pH=8.0) is added to dissolve the DNA precipitate to prepare a DNA storage solution.
(8) The DNA storage solution is diluted to 20-30 ng/μL with ultrapure water as a working solution for later use.

### 3. Identification and Genetic Analysis of Powdery Mildew Resistance in Wheat Seedling Stage

The identification of powdery mildew resistance in a wheat seedling stage is completed in an artificial climate chamber. The resistance parent Triticum dicoccoides DR739, the susceptible parent Triticum turgidum Langdon, the hybrid F₁, the F₂ population and the F_{2:3} families are planted in a 128-hole tray (2×2 cm), 5 seeds are sown in each hole, 20 seeds of each of the patents and F₁ are identified, 20 seeds of each F_{2:3} family are sown, and a susceptible control Mingxian 169 is sown randomly and labeled. After sowing, growth conditions are controlled as a 14 hours light/10 hours darkness cycle, a temperature of 20° C., and relative humidity of 30-40%. When the seedlings grow to a one leaf stage, and are inoculated with a wheat powdery mildew strain E09. The conditions for the first 24 hours after inoculation are controlled as follows: darkness, a temperature of 20° C., and relative humidity of 100%. Then, the conditions are controlled as follows: a 14 hours light/10 hours darkness cycle, a temperature of 18-22° C., and relative humidity of 100%. When the first leaf of the susceptible control Mingxian 169 is fully infected, phenotypes are recorded according to the standards of grades 0-4, where grades 0-2 are regarded as resistance grades, and grades 3 and 4 are regarded as susceptible grades.

Investigation results show that the Triticum dicoccoides DR739 is immune to the strain E09, and the Triticum turgidum Langdon (LDN) is highly susceptible to the strain E09. Hybrid F₁ between the Triticum dicoccoides DR739 and the Triticum turgidum Langdon (LDN) shows resistance. 109 of 141 F₂ plants show resistance, and 32 show susceptibility, which accords with a segregation ratio of resistance to susceptibility of 3:1. Among 141 F_{2:3} families, 34 are homozygous for resistance, 75 are heterozygous, 32 are homozygous for susceptibility, and the segregation ratio of homozygous resistance:heterozygosis:homozygous susceptibility is 1:2:1. Therefore, the resistance of the Triticum dicoccoides DR739 to the strain E09 is controlled by a single dominant gene, named gene PmDR739.

### 4. Initial Mapping of Molecular Marker of Gene PmDR739

According to phenotypic identification results, 20 homozygous resistance families and 20 homozygous susceptible families are selected to construct a resistance pool and a susceptible pool respectively. Polymorphism detection is performed on the Triticum dicoccoides DR739, the Triticum turgidum Langdon, the resistance pool and the susceptible pool by using genome-wide evenly distributed molecular markers. 10 pairs of markers show consistent polymorphism in the resistance and susceptible parents and the resistance and susceptible pools. Then, 141 F_{2:3} families of Triticum dicoccoides DR739×Triticum turgidum Langdon are genotyped by using these markers, and the gene PmDR739 is initially mapped at a terminal of the wheat chromosome 2AL.

### 5. Development of Markers Closely Linked to Gene PmDR739

According to sequence information of the reference genome (Wild Emmer v2.0) of the Triticum dicoccoides, simple sequence repeat (SSR) markers are designed through software primer5.0. The F_{2:3} families of Triticum dicoccoides DR739×Triticum turgidum Langdon are genotyped, the SSR marker HENU686 closely linked to the gene PmDR739 is obtained, and a genetic distance is only 0.85 cM.

A primer of the molecular marker HENU686 includes a forward primer and a reverse primer:
nucleotide sequence of forward primer HENU686-F:

5′-CGGTGTCCTGGTGTTCTTGTC-3′, as shown in SEQ
ID NO: 1;

nucleotide sequence of reverse primer HENU686-R:

5′-TGGAGTATTTGGGTGCATTTC-3′, as shown in SEQ
ID NO: 2.

A PCR amplification system is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification is: predenaturation for 3 minutes at 94° C., denaturation for 15 seconds at 94° C., annealing for 20 seconds at 55° C., extension for 40 seconds, and 30 cycles; extension for 10 minutes at 72° C.; and preservation at 4° C.

A procedure of electrophoresis of an amplification product is: electrophoresis is performed on a non-denaturing polyacrylamide gel with the mass volume percentage concentration of 8%, after the amplification product is mixed with 2 μL of 6×sample loading buffer, 2 μL of the mixture is taken for spot sampling, electrophoresis is performed under constant pressure of 180 V for 2.5-3 hours, and photographing is performed after silver nitrate staining.

Amplification results show: two specific bands of 280 bp and 370 bp are simultaneously amplified, and wheat to be detected is indicated to carry the powdery mildew resistance gene PmDR739 of Triticum dicoccoides; and two specific bands of 280 bp and 370 bp fail to be simultaneously amplified, and the wheat to be detected is indicated not to carry the powdery mildew resistance gene PmDR739 of Triticum dicoccoides.

Embodiment 2 Application of molecular marker HENU686 primer of powdery mildew resistance gene PmDR739 of wheat

Triticum dicoccoides DR739, Triticum turgidum Langdon and materials in F_{2:3} families formed by crossing the Triticum dicoccoides DR739 with the Triticum turgidum Langdon are detected by the molecular marker HENU686 of the powdery mildew resistance gene PmDR739 of wheat, and a sample DNA extraction method is the same as that in Embodiment 1.

Samples to be detected: Triticum dicoccoides DR739, Triticum turgidum Langdon and F_{2:3} families formed by crossing the Triticum dicoccoides DR739 with the Triticum turgidum Langdon

Genomic DNAs of the above materials are extracted as a template of PCR amplification, and the amplification is performed through the primer of the molecular marker HENU686 developed by the present disclosure.

Nucleotide sequence of forward primer:
HENU686-F:
5′-CGGTGTCCTGGTGTTCTTGTC-3′, as shown in SEQ
ID NO: 1,
and
nucleotide sequence of reverse primer:
HENU686-R:
5′-TGGAGTATTTGGGTGCATTTC-3′, as shown in SEQ
ID NO: 2.

A PCR amplification system is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification is: predenaturation for 3 minutes at 94° C., denaturation for 15 seconds at 94° C., annealing for 20 seconds at 55° C., extension for 40 seconds, and 30 cycles; extension for 10 minutes at 72° C.; and preservation at 4° C.

A procedure of electrophoretic segregation of an amplification product is: electrophoresis is performed on a non-denaturing polyacrylamide gel with the mass volume percentage concentration of 8%, after the amplification product is mixed with 2 μL of 6×sample loading buffer, 2 μL of the mixture is taken for spot sampling, electrophoresis is performed under constant pressure of 180 V for 2.5-3 hours, and photographing is performed after silver nitrate staining.

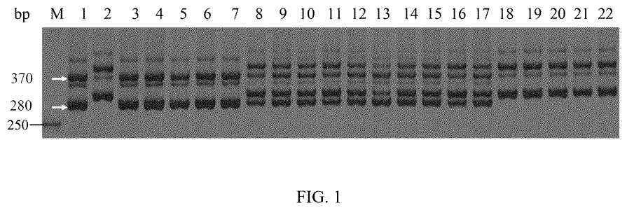

Detection results of the primer of the molecular marker HENU686 in the materials to be detected are shown in FIG. 1. FIG. 1 shows some amplification results of the primer of the molecular marker HENU686 in the Triticum dicoccoides DR739, the Triticum turgidum Langdon as well as F_{2:3} families derived from Triticum dicoccoides DR739×Triticum turgidum Langdon. In the figure, M: DL2000; 1: Triticum dicoccoides DR739 (powdery mildew resistance variety carrying the gene PmDR739); 2: Triticum turgidum Langdon (powdery mildew susceptible variety); 3-22: F_{2:3} families formed by crossing the Triticum dicoccoides DR739 with the Triticum turgidum Langdon, where 3-7: F_{2:3} family homozygous for resistance, 8-17: F_{2:3} family for resistance and susceptibility segregation, and 18-22: F_{2:3} family homozygous for susceptibility; and bands indicated by the two white arrows are two specific bands capable of tracing the gene PmDR739. Amplification results show that the varieties with two specific bands of 280 bp and 370 bp amplified simultaneously are varieties of the resistance parent Triticum dicoccoides DR739 and resistance families, and the varieties without such target bands are varieties of the susceptible parent Triticum turgidum Langdon and other susceptible families.

The gene PmDR739 originated from the Triticum dicoccoides DR739, and mapping and map-based cloning of the gene has not been reported yet. The molecular marker HENU686 and the primer provided by the present disclosure are used for detecting a large genetic mapping population, which contributes to fine mapping and map-based cloning of the gene PmDR739, and has very important significance for efficient transformation of the gene PmDR739 and in-depth analysis of a disease resistance mechanism.

The above embodiments are optimized implementation plans of the present disclosure, and are only used for illustrating the present disclosure, not limiting the present disclosure. All modifications or equivalent substitutions made by those skilled in the art without departing from the purpose and principle of the implementation plans of the present disclosure fall within the scope claimed to be protected by the present disclosure.

What is claimed is:

1. A primer for amplifying a closely linked molecular marker of a powdery mildew resistance gene PmDR739 of Triticum dicoccoides, comprising a forward primer HENU686-F and a reverse primer HENU686-R, wherein the nucleotide sequence of the forward primer HENU686-F is shown as SEQ ID NO:1, and the nucleotide sequence of the reverse primer HENU686-R is shown as SEQ ID NO:2.

2. An application of the primer for amplifying a closely linked molecular marker of a powdery mildew resistance gene PmDR739 of Triticum dicoccoides according to claim 1 in detection and identification of the gene PmDR739, auxiliary identification of a powdery mildew resistance trait of wheat, and molecular marker-assisted selection breeding.

3. A method for detecting whether a wheat sample carries a powdery mildew resistance gene PmDR739 of Triticum dicoccoides, comprising the following steps:
(1) extracting a genomic deoxyribonucleic acid (DNA) of a wheat sample to be detected;
(2) performing polymerase chain reaction (PCR) amplification on the genomic DNA of the wheat sample to be detected by utilizing a molecular marker primer to obtain an amplification product; and
(3) performing electrophoresis and detection on the amplification product, wherein if two specific bands of 280 bp and 370 bp are capable of being simultaneously amplified, the wheat sample to be detected is indicated to carry the powdery mildew resistance gene PmDR739 of Triticum dicoccoides, otherwise, the wheat sample to be detected is indicated not to carry the powdery mildew resistance gene PmDR739 of Triticum dicoccoides.

4. The method according to claim 3, wherein the molecular marker primer in step (2) comprises a forward primer HENU686-F and a reverse primer HENU686-R, the nucleotide sequence of the forward primer HENU686-F is shown as SEQ ID NO:1, and the nucleotide sequence of the reverse primer HENU686-R is shown as SEQ ID NO:2.

5. The method according to claim 3, wherein a PCR amplification system in step (2) is 10 μL, comprising: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

6. The method according to claim 3, wherein a procedure of the PCR amplification in step (2) is: performing predenaturation for 3 minutes at 94° C., performing denaturation for 15 seconds at 94° C., performing annealing for 20 seconds at 55° C., performing extension for 40 seconds, and performing 30 cycles; performing extension for 10 minutes at 72° C.; and performing preservation at 4° C.
